(19) United States
(12) Patent Application Publication
(10) Pub. No.: US 2021/0151133 A1
(43) Pub. Date: May 20, 2021

(54) Method For Designing For Temperature Sensitivity Of Hydration Of Cement Slurry

(71) Applicant: Halliburton Energy Services, Inc., Houston, TX (US)

(72) Inventors: John Paul Bir Singh, Kingwood, TX (US); Thomas Jason Pisklak, Cypress, TX (US); Ronnie Glen Morgan, Waurika, OK (US); Krishna Babu Yerubandi, Houston, TX (US)

(73) Assignee: Halliburton Energy Services, Inc., Houston, TX (US)

(21) Appl. No.: 16/637,244

(22) PCT Filed: Apr. 5, 2019
(86) PCT No.: PCT/US2019/026176

§ 371 Date: Feb. 6, 2020

(51) Int. Cl. G16C 60/00; E21B 33/14; G16C 20/30; G16C 20/50; G06F 30/20

(52) U.S. Cl. CPC G16C 60/00; E21B 33/14; G16C 20/30; G06F 2119/08; G06F 30/20; E21B 2200/20; G16C 20/50

(57) ABSTRACT

A method may include: providing a model of cement temperature sensitivity; designing a cement composition, based at least partially on the model of cement temperature sensitivity; and preparing the cement composition.

### BACKGROUND

In well cementing, such as well construction and remedial cementing, cement slurries are commonly utilized. Cement slurries may be used in a variety of subterranean applications. For example, in subterranean well construction, a pipe string (e.g., casing, liners, expandable tubulars, etc.) may be run into a well bore and cemented in place. The process of cementing the pipe string in place is commonly referred to as “primary cementing.” In a typical primary cementing method, a cement slurry may be pumped into an annulus between the walls of the well bore and the exterior surface of the pipe string disposed therein. The cement slurry may set in the annular space, thereby forming an annular sheath of hardened, substantially impermeable cement (i.e., a cement sheath) that may support and position the pipe string in the well bore and may bond the exterior surface of the pipe string to the subterranean formation. Among other things, the cement sheath surrounding the pipe string functions to prevent the migration of fluids in the annulus, as well as protecting the pipe string from corrosion. Cement slurries also may be used in remedial cementing methods, for example, to seal cracks or holes in pipe strings or cement sheaths, to seal highly permeable formation zones or fractures, to place a cement plug, and the like.

A particular challenge in well cementing is the development of satisfactory mechanical properties in a cement slurry within a reasonable time period after placement in the subterranean formation. Oftentimes several cement slurries with varying additives are tested to see if they meet the material engineering requirements for a particular well. The process of selecting the components of the cement slurry are usually done by a best guess approach by utilizing previous slurries and modifying them until a satisfactory solution is reached. The process may be time consuming and the resulting slurry may be complex. Furthermore, the cement components available in any one particular region may vary in slurry from those of another region thereby further complicating the process of selecting a correct slurry.

### BRIEF DESCRIPTION OF THE DRAWINGS

FIGS. 1a and 1b are normalized graphs depicting time-dependent extent of hydration for a cement slurry with a low activation and a cement slurry with a high activation energy.

FIG. 2 is a graph of measured activation energy and predicted activation energy.

FIG. 3 is a graph of measured activation energy and predicted activation energy.

FIG. 4 illustrates surface equipment that may be used in the placement of a cement slurry.

FIG. 5 illustrates a cement slurry being placed in a wellbore.

FIG. 6 is a schematic illustration of an example of an information handling system.

FIG. 7 illustrates additional detail of an information handling system.

### DETAILED DESCRIPTION

The present disclosure may generally relate to cementing methods and systems. More particularly, embodiments may be directed to designing a cement slurry using a model of cement temperature sensitivity.

One common type of cement used in wellbore and surface cementing is Portland cement. Portland cement may undergo a hydration reaction to produce a hardened mass. The various oxides, such as calcium oxides, and silicates present in the Portland cement may undergo a crosslinking reaction induced by water to produce a hydrated cement paste which may then set to form the hardened mass. A Portland cement slurry including Portland cement and water may have four distinct phases of hydration, sometimes referred to herein as cementitious reactions. The first stage, termed the pre-induction period may be characterized by rapid heat evolution and subsequent temperature rise from the heat evolution. During the pre-induction period, calcium and hydroxyl ions may be released into the slurry. The second stage, termed the induction period, may be characterized by a period of extended time where temperature of the slurry decreases and there is little to no compressive strength development. During the induction period, the Portland cement slurry may remain in a pumpable fluid state such that the Portland cement slurry may be placed in a wellbore or other location of interest. The induction period may see continued dissolution of calcium oxide into the slurry and continued reactions which may not significantly contribute to compressive strength. While the exact identity and molecular nature of the reactions that are ongoing during the induction period may not be precisely known, one of ordinary skill in the art will understand that there is an activation energy associated with the induction period that may be related to the chemical identity and concertation of species present in the slurry. The activation energy may be the amount of energy required to overcome an activation barrier to allow the slurry to progress to the rapid strength development stage. The rapid strength development period may be characterized by a rapid increase in reactions that contribute to compressive strength development of the cement slurry. During the rapid strength development period the cement slurry transitions from a pumpable slurry to a slurry that becomes steadily more viscous and to a point where the slurry is considered unpumpable. Usually, a slurry is considered unpumpable when the slurry reaches 70 Bc (Bearden units of consistency). However, depending on the application, a cement slurry may be considered unpumpable at a higher or lower consistency. The slurry may continue to develop viscosity and eventually set to from a cohesive hardened mass. A cement slurry may have a particular target 24 hour compressive strength which may be the compressive strength achieved after a 24 hour period after mixing dry cement with water. A fourth stage, referred to as the long term compressive strength development stage may start after the cement slurry has set to form the hardened mass. The long term compressive strength development stage may be characterized by continued hydration of the oxides and silicates which may contribute to an increase in compressive strength over a period of days to years.

In general, Portland cement may include 5 main minerals and, in some examples, other minerals that make up a minor portion of the Portland cement. The minerals may include dicalcium silicate (C₂S), tricalcium silicate (C₃S), tricalcium aluminate (C₃A), tetra calcium alumino ferrite (C₄AF), and gypsum. As one of ordinary skill in the art will understand, each of the main cement minerals may hydrate at different rates and may form different solid phases when they hydrate. Rate of hydration of each mineral in Portland cement may be dependent on many factors including temperature of the cement slurry. In general, a higher temperature may cause the hydration reactions to occur at a faster rate leading to a shorter induction time and more rapid gain of compressive strength. Many of the reactions that take place during hydration of Portland cement may be exothermic such that the temperature of the cement slurry increases during the pre-induction period and the rapid strength development stage. The temperature rise may lead to even faster cementitious reactions and more temperature rise until the cementitious reactions taper off and the Portland cement transitions into the long-term compressive strength development stage. Additionally, when the cement slurry is pumped into a wellbore, elevated temperatures in the wellbore may heat the cement slurry causing the cementitious reactions to proceed at an increased rate as compared to cement slurries that are used in surface cementing applications. Although only pure Portland cement slurries have been discussed until this point, one of ordinary skill in the art will readily recognize other that a cement slurry may include many different cementitious components such as Portland cement, pozzolans, clays, silica, fly ash, slag, and many other components one of ordinary skill in the art will readily recognize. Each cementitious component may include cementitious minerals. The cementitious reactions that occur in any one cement slurry may be a function of the slurry of the cement slurry and more particularly to the individual minerals and concentrations thereof that are present in the cement slurry.

There may be many potential reactions that occur during hydration of a cement slurry owning to the multitude of minerals that may be present in the cement slurry. It may be difficult to model each reaction for a plurality of reasons, including, but not limited to, that not all cementitious reaction may be known, measuring or predicting concentrations of intermediate species may be difficult, and kinetic rate parameters of individual reactions may not be known. A model that may represent the extent of hydration without needing to account for each individual cementitious reaction is illustrated in Equation 1.

$$H = {H}_{u} {e}^{- {( \frac{τ}{{t}_{e}} )}^{β}} \qquad ( 1 )$$

Rather than calculate each individual rate constant and derive concentrations of intermediate hydration species, Equation 1 shows the extent of hydration H as a function of ultimate extent of hydration Hᵤ, effective time tₑ, and kinetic rate parameters τ and β. Extent of hydration is directly correlated to the extent of strength development of a cement slurry. An extent of hydration may have any scale. In an example, the extent of hydration may be from 0 to 1, where 0 corresponds to no hydration and 1 corresponding to full hydration of ultimate extent of hydration. When the cement slurry is completely non-hydrated, the cement slurry will have no compressive strength and when the cement slurry is completely hydrated, the cement slurry will have the ultimate compressive strength. Effective time is the temperature correction applied to the cement slurry to account for the temperature dependency of the cementitious reactions. The effective time may be derived from Equation 2.

$$\frac{\partial {t}_{e}}{\partial t} = exp ( \frac{E}{R} ( \frac{1}{{T}_{ref}} - \frac{1}{T} ) ) \qquad ( 2 )$$

In Equation 2, E is the activation energy, R is the gas constant, T_{ref} is the reference temperature, and T is the current temperature. Equation 2 allows the temperature effects on cement hydration to be calculated in terms of effective time. When the temperature is at the reference temperature, the effective time is the same as real time. At temperatures that are greater than the reference temperature, the cementitious reactions may proceed at a faster rate. At temperatures that are less than the reference temperature, the cementitious reaction may proceed at a slower rate. One reason for slower reactions at lower temperature may be that total available energy at lower temperatures may be less than at higher temperatures. Lower available energy may mean that less of the cement slurry is at or above the activation energy.

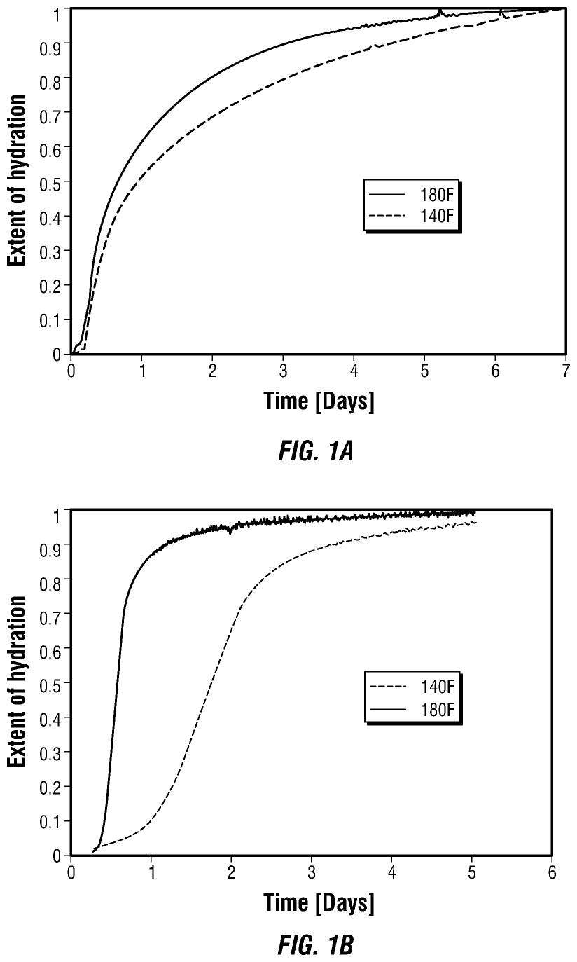

FIGS. 1a and 1b are normalized graphs depicting time-dependent extent of hydration for a cement slurry with a low activation and a cement slurry with a high activation energy where each cement slurry was hydrated at a high and a low temperature. In FIG. 1a, time dependent hydration of a low activation energy cement is shown at 180° F. (82.2° C.) and 140° F. (60° C.). It can be observed that the extent of hydration is minimally changed between the lower and higher temperature which may indicate that there is enough energy available at the lower temperature to hydrate the cement slurry. In FIG. 1b, time dependent hydration of a high activation energy cement is shown at 180° F. (82.2° C.) and 140° F. (60° C.). It can be observed that the extent of hydration is severely affected by the temperature as the slurry hydrated at the lower temperature takes about 4 days to reach 90% hydration versus the slurry hydrated at the higher temperature takes about 1 day to reach 90% hydration. Although both the low activation energy and high activation energy cement slurry hydration are slowed by a lower hydration temperature, the high activation energy cement slurry experiences a large decrease in hydration rate as compared to the low activation energy cement slurry at the lower hydration temperature.

There may be many cementitious materials available for use in a cement slurry, each of which may have an associated activation energy, and workable temperature range where the cementitious material may be appropriate for use. For example, cements blended with fly ash may have a high activation energy, and therefore may have slower kinetics at relatively lower temperatures as compared to a neat Portland cement. Fly ash blended cements may be preferred to be used at higher temperatures due to the slower kinetics and as such may be unfavored at temperatures below about 140° F. (60° C.). Similarly, use of Type I/II or class A cement may be unfavored at relatively higher temperatures as they may have lower activation energies and thus be highly reactive as compared to class G or class H cements at elevated temperatures. In some cement slurries, accelerators and retarders may be added to the cement slurry to tune the set time of the cement slurry such that the cement slurry sets within a desired timeframe. While cement retarders and accelerators may provide certain benefits in a cement slurry, one of ordinary skill in the art will readily recognize that there may also be disadvantages to including accelerators and retarders in cements. Some disadvantages may include, but not limited to, complexity of slurry design, early setting, gelation, gas migration, and chemical incompatibility with other cement additives.

The methods described herein may allow one of ordinary skill in the art, with the benefit of this disclosure, to design a cement slurry with a desired temperature sensitivity and performance characteristic. Designing a cement slurry may include selecting concentrations and identities of components of a cement the cement slurries. The cement slurries may require fewer cement additives and/or smaller concentrations of cement additives to achieve the performance characteristic as compared to a cement slurry that was not designed by the methods disclosed herein. In some examples, the performance characteristic may be temperature sensitivity. Using the methods described herein, one of ordinary skill in the art may be able to design a cement slurry that exhibits a tuned temperature sensitivity so that the cement slurry may hydrate and set to form a hardened mass within a desired time period. The methods described herein may also allow one of ordinary skill in the art to design a wellbore cement slurry wherein a column of cement placed in a wellbore sets concurrently. The methods described herein may also allow one of ordinary skill in the art to design a wellbore cementing slurry which exhibits reduced or increased temperature sensitivity such available cementitious materials may be utilized in a greater range of wellbore temperatures.

As shown in Equations 1 and 2, the variables which control the rate of hydration may be activation energy and time. In general, the temperature which the cement slurry will be placed at is known or defined and adjusting the rate of hydration based on the temperature may not be possible. For example, in wellbore cementing applications, a well log may be taken during drilling or during an open hole logging operation such that the temperature at each point in the wellbore is known. Additionally, wellbores drilled in the same geographic area or in the same subterranean formation may exhibit similar temperature profiles such that the temperature profile of one wellbore may be used to predict the temperature profile of another wellbore drilled in the same geographic area or same subterranean formation if a wellbore logging operation is not performed. While there may be techniques that involve cooling a wellbore before placing a cement slurry therein, the techniques may be expensive, time consuming, and may still result in a wellbore that is not temperature controlled. In surface cementing applications, there may be more techniques available to control the hydration temperature, but they again may be time consuming and expensive. In surface cementing applications, the hydration temperature may be the ambient temperature, for example. Those of ordinary skill in the art would be able to measure or predict the temperature a cement slurry will set at.

Since the temperature where the cement slurry will be placed at may not be a controllable parameter, one method to control the rate, and time dependent extent of hydration may be to adjust the activation energy of a cement slurry. The activation energy of the cement slurry may be adjusted by adjusting the relative amounts of the cementitious components in the cement slurry, for example. Each component in the cement slurry may include various minerals that together provide the activation energy for the particular cementitious component. However, the effects on activation energy of adding more or less of a particular cementitious component may not be readily predicted ahead of time due to the intermediate products previously discussed. A method to determine the activation energy without modeling each individual reaction will now be discussed.

Hydration of a cement slurry releases heat which may increase the temperature of the cement slurry. The heat release may allow for calorimetry studies to be performed on the cement slurry and the calorimetry data may be used to calculate an activation energy for a cementitious component. One of ordinary skill in the art, with the benefit of this disclosure would readily recognize the techniques appropriate for determining an activation energy from calorimetric data. Calorimetry data may also be used to determine temperature sensitivity of each component in a cement slurry by, for example, varying concentrations of each component in a cement slurry and varying the temperatures at which the cement composition sets. A regression analysis may be performed on the data generated to build a regression model of temperature sensitivity. Furthermore, physicochemical tests may be performed on each cementitious component to generate physicochemical data about each cementitious component. Some examples of physicochemical data may include, but are not limited to, concentration and identity of minerals present in the cementitious component and surface area, for example.

Once the activation energy for each cement slurry is determined, a non-linear regression analysis may be performed on the activation energy data and the physicochemical data to estimate the relationships between the activation energy and physicochemical properties of cement slurries. The regression analysis may output a regression model which may be used to approximate the effects of the physicochemical data, such as mineralogical slurry, on the activation energy. In some examples, the regression analysis may be linear regression, polynomial regression, multivariate linear regression, logarithmic, power, exponential, step-wise, or any other regression method. One of ordinary skill in the art, with the benefit of this disclosure, would be able to select an appropriate regression analysis for a particular application. The regression model may be used to calculate the activation energy of an individual cement component or a combination of cement components. As previously discussed, as the cement slurry hydrates, the minerals in the cement slurry may dissolve, react, and form intermediate products. The intermediate products may be difficult to directly observe or individually model owing to the complex nature of synergistic interactions between intermediate products. The chemical behavior of cementitious minerals has been studied by synthesizing a cementitious mineral in its pure form and hydrating it under controlled conditions. During the actual cement hydration process all the minerals dissolve into the same pore solution, and thus the solid hydration products are associated with the pore solution as a whole and the interaction between the dissolved minerals therein rather than a particular cementitious mineral. A regression model derived from calorimetric and physicochemical data may indirectly approximate the interactions between the minerals making up the cement slurry and the activation energy of the cement slurry without the need to individually model all reactions. In the regression model, the activation energy of the cement slurry may be a dependent variable while the physicochemical properties of the cement slurry may be the independent variables. In addition to calculating the activation energy of a cementitious component or blends of cementitious components, the regression model may also allow one of ordinary skill in the art to design a cement slurry with a target activation energy. Interaction effects between different mineral species can also be studied using statistical techniques such as by analysis of variance (ANOVA) and others, for example.

Equation 3 shows a generalized regression model for any cement slurry which includes components of C₂S, C₃S, C₄AF, C₃A, CaSO₄, and gypsum. The generalized regression model also contains average particle size, BET surface area, and specific surface area variables. As one of ordinary skill in the art, with the benefit of this disclosure will appreciate, a regression model may include any number of parameters not specifically listed herein.

Eₐ=f(particle size,BET,SSA,C₂S,C₃S,C₄AF,C₃A,CaSO₄,gypsum) (3)

Additionally, the regression model may contain more independent variables, or fewer independent variables depending on the desired accuracy of the regression model. Furthermore, there may be some independent variables which may have little impact on the activation energy and therefore may be excluded from the regression model. The regression model may also include interaction parameters between the independent variables. Equations 4 and 5 illustrate how the regression model is generated and how the regression model may be used. The regression model may take physicochemical data of a cement slurry as an input and output an estimate of the activation energy of the cement slurry. The regression model may also be used to design a cement slurry with a target activation energy by providing the target activation energy and using an iterative method to find a combination of independent variables which results in an output of the target activation energy from the regression model. Some iterative methods may include, but are not limited to, unconstrained multivariate optimization, constrained multivariate optimization, unconstrained optimization, and constrained optimization, for example.

Data→Regression Model (4)

Physiochemical Data⇄Regression Model⇄Activation Energy (5)

The regression model of activation energy may also be combined with other models of cement slurries such as models of compressive strength, mixability, and induction time models, for example, to calculate a cement slurry that satisfies multiple requirements. For example, a constrained multivariate optimization of a cement slurry may include a compressive strength requirement and an activation energy requirement. A constrained multivariate optimization may be used to calculate a cement slurry which satisfies the activation energy requirement and compressive strength requirement.

To better illustrate how the foregoing discussion may be applied to a Portland cement slurry, an example of deriving a regression model of activation energy will be presented. Physicochemical analysis of a plurality of cement slurries was performed to study the mineralogical makeup and surface areas of each of the cement slurries. A calorimetry study was also performed on the plurality of cement slurries to calculate the activation energy of each cement slurries. Table 1 lists the results of the physicochemical study and Table 2 lists the results from the calorimetry study.

TABLE 1

| Cement Type | Class A | Class C | Class G | Class H (1) | Class H (2) | Class H (3) | Type I | Type III |
|---|---|---|---|---|---|---|---|---|
| C3S | 55.3 | 70.47 | 48.02 | 47.11 | 45.66 | 59.66 | 59.5 | 16.69 |
| C2S | 17.49 | 3.41 | 26.03 | 27.18 | 28.6 | 18.2 | 13.07 | 23.39 |
| C34AF | 8.54 | 11.13 | 17.56 | 17.67 | 17.44 | 14.16 | 10.51 | 3.83 |
| C3A | 10.02 | 5.93 | 2.29 | 0 | 0 | 0.41 | 6.8 | 12.74 |
| gypsum | 5.22 | 5.63 | 3.98 | 4.63 | 4.7 | 4.94 | 5.71 | 5.88 |
| surface area | 280 | 418 | 329 | 254 | 285 | 303 | 391 | 548 |
| (cm\hat{ }2/g) | | | | | | | | |

TABLE 2

| | Activation |
|---|---|
| Cement Type | Energy (j/mol) |
| Class A | 41163 |
| Class C | 40493 |
| Class G | 36333 |
| Class H (1) | 36505 |
| Class H (2) | 38811 |
| Class H (3) | 41987 |
| Type I | 41430 |
| Type III | 43599 |

A regression model was calculated from the data in Tables 1 and 2 and is illustrated in Equation 6. In equation 6, A, B, and C are constants determined during the calculation of the regression model. Ea is the activation energy of the Portland cement blend.

Eₐ=A−B*C₄AF (%)−C*C₃A (%) (6)

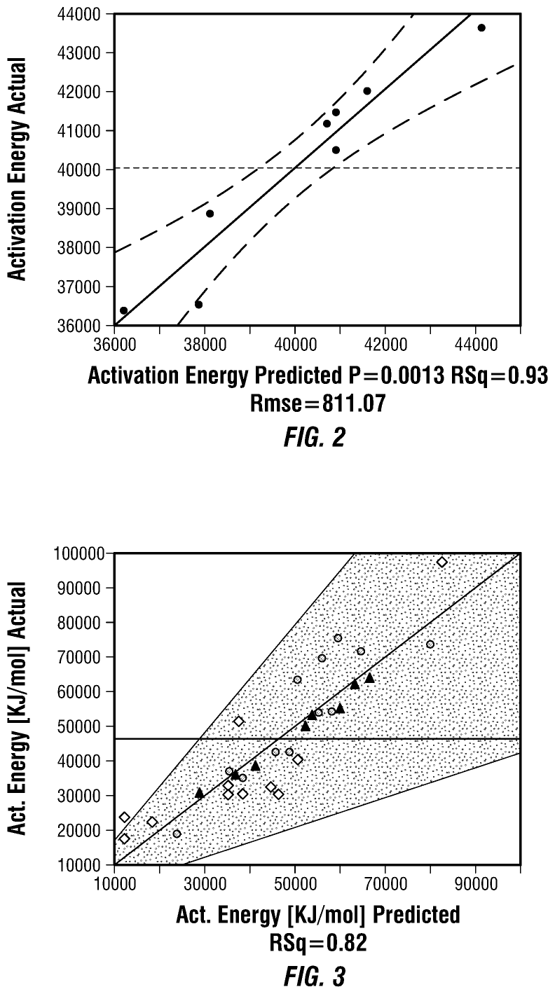

FIG. 2 illustrates the result of graphing the measured activation energy of Table 2 and the predicted activation energy from equation 6 combined with the physicochemical data of Table 1. It can be observed that the coefficient of determination is 0.93.

A second test was performed with thirteen hydraulic and siliceous materials including Portland cements, natural glasses, natural glasses, cement kiln dust, fly ash, silica fume, and blast furnace slag. Thirty-eight blends of the hydraulic and siliceous materials were prepared at various densities and temperatures. Physicochemical analysis and activation energy studies were performed as described above. A regression analysis was performed on the physicochemical and activation energy data to generate a regression model in the form of Equation 7.

Eₐ=Eₚ*Portland+E_{VG1}*NaturalGlass+E_{VG2}*NaturalGlass2+E_{CKD}*CKD+E_{FA}*Fly Ash+E_{SF}*SilicaFume+E_{BFS}*BFS (7)

Where Eₚ, E_{FA}, E_{SF}, E_{BFS}, E_{VG1}, E_{VG2}, E_{CKD}, E_{SF} are constants and Portland, Natural Glass, Natural Glass2, CKD, Fly Ash, SilicaFume, and BFS are concentrations. The predicted activation energy from the regression model was compared to the measured activation energy and plotted in FIG. 3. It can be observed that the coefficient of determination for the model is 0.82.

A method of testing a cement slurry using activation energy will now be described. The method may include surveying cementitious components available at a location, obtaining samples of the cementitious components, and physiochemically characterizing the cementitious components. The results of the physicochemical testing may be used in a regression model of activation energy to predict the activation energy of a blend including at least some of the cementitious components. A downhole temperature and pressure log for a wellbore to be cemented may be provided or other techniques previously described to predict pressure and temperature may be employed. In particular, the bottom hole temperature and temperature at top of cement (TOC) may be provided. In some examples, a compressive strength time requirement may also be provided. Some wellbore cementing operations, whether by regulatory requirement, customer requirement, or best practice, may require a particular compressive strength development in a specified amount of time. Sometimes the compressive strength time requirement may be referred to as time to 50 psi (345 kPa) or time to 500 psi (3450 kPa). Although only some compressive strength time requirements are given herein, the compressive strength time requirement may have any value at any time. A proposed cement slurry may be provided wherein the proposed cement blend includes at least some of the cementitious components. The cement slurry may be prepared, and the extent of hydration may be tested at the highest expected bottom hole temperature. The activation energy for the proposed cement blend may be calculated from Equations 1 and 2 using the extent of hydration data. The time dependent compressive strength may then be predicted using a regression model derived from the extent of hydration which is directly correlated to the compressive strength development of the cement blend. If the cement blend does not meet or exceed the compressive strength time requirement, the proposed cement slurry may be modified, and the method described above may be repeated for the modified cement slurry. The cement slurry may be iterated until the cement slurry meets or exceeds the compressive strength time requirement.

Another method may include designing a cement slurry using a regression model of activation energy to design a cement that will have a target compressive strength sometimes referred to a design parameter at a chosen time. A pumping rate for introducing the cement slurry and conduit dimensions for the conduit to be cemented may be provided. As previously discussed, the temperature along a wellbore may be known from logging or other techniques. Heat transfer from the formation into the wellbore and heat generated by the cement slurry may be modeled such that a time dependent temperature of a cement slurry introduced into the wellbore may be approximated at each point in the wellbore. Equations 1 and 2 along with the regression model of activation energy and the heat transfer model may be used to formulate a cement slurry that will set at any desired time in the wellbore. An iterative technique may be used with the regression model to generate a cement slurry and associated activation energy. The cement slurry and activation energy may be combined with Equations 1, 2, and the heat transfer model to estimate the compressive strength of the cement slurry at any time, and therefore any point in the wellbore. In an example, a compressive strength time requirement may be provided. The compressive strength time requirement may be compared to the compressive strength estimated by the above method. If the compressive strength time requirement is greater than the estimated compressive strength, the iteration may be continued until a slurry that satisfies the compressive strength time requirement is reached. Any of the above mentioned techniques may be combined with other models such as compressive strength development models, induction time models, and other models in a multivariate optimization such that the cement slurry generated may satisfy multiple objectives.

The methods described above may also allow for designing cement slurries for temperature sensitivity. In some applications, the wellbore to be cemented may be relatively cold or relatively hot which may require the use of cement additives to accelerate or retard the cement slurry during setting. The methods described above may allow a cement slurry with reduced cement additives to be generated which still meets or exceeds a time dependent compressive strength for relatively cold or relatively hot wellbores.

Although only some regression models have been illustrated herein, one of ordinary skill in the art, with the benefit of this disclosure would be able to derive other forms of the equations herein without deviating from this disclosure. In addition, the techniques and equations described herein may also be applied to surface cementing application such as construction cementing.

Measuring physicochemical properties of each selected cement component may include many laboratory techniques and procedures including, but not limited to, microscopy, spectroscopy, x-ray diffraction, x-ray fluorescence, particle size analysis, water requirement analysis, scanning electron microscopy, energy-dispersive X-ray spectroscopy, surface area, specific gravity analysis, thermogravimetric analysis, morphology analysis, infrared spectroscopy, ultraviolet-visible spectroscopy, mass spectroscopy, secondary ion mass spectrometry, electron energy mass spectrometry, dispersive x-ray spectroscopy, auger electron spectroscopy, inductively coupled plasma analysis, thermal ionization mass spectroscopy, glow discharge mass spectroscopy x-ray photoelectron spectroscopy, mechanical property testing, Young's Modulus testing, rheological properties, Poisson's Ratio. One or more of the proceeding tests may be consider API tests, as set forth in the API recommended practice for testing well cements (published as ANSI/API recommended practice 10B-2). Additional API tests not specifically listed above may also be used for the measurements. The physical and chemical properties may be measured for a group of cement components. Two or more of the cement components measured may be different types of cement components (e.g., natural glass, CKD, fly ash, etc.). Two or more of the cement components may be the same type but from different sources (e.g., natural glass from source 1, natural glass from source 2, etc.).

X-ray powder diffraction is one analysis technique that may be used for measuring the physical and chemical properties of the cement components. X-ray powder diffraction is a technique of exposing a sample to x-rays, neutrons, or electrons and measuring the amount of inter-atomic-diffraction. The sample acts a diffraction grating thereby producing a differing signal at different angles. The typical properties that may be measured are the phase identification for the identification and characterization of a crystalline solid. Other properties may be crystallinity, lattice parameters, expansion tensors, bulk modulus, and phase transitions.

X-ray fluorescence is another analysis technique that may be used for measuring the physical and chemical properties of the cement components. X-ray fluorescence may use short wave x-rays to ionize atoms in a sample thereby causing them to fluoresce at certain characteristic wavelengths. The characteristic radiation released by a sample may allow accurate identification of the component atoms in the sample as well as their relative amounts.

Particle size analysis is another analysis technique that may be used for measuring the physical and chemical properties of the cement components. Particle size analysis may be accomplished through analysis by various laboratory techniques including but not limited to laser diffraction, dynamic light scattering, static image analysis, and dynamic image analysis. Particle size analysis may also provide information about the morphology of a particular sample. Morphology may include parameters such as sphericity and roundness as well as the general shape of a particle such as disk, spheroid, blade, or roller. With a knowledge of the morphology and particle size, the average surface area and volume may be estimated. Surface area and volume may be important in determining the water requirement as well as reactivity. In general, a relatively smaller particle size may react more quickly than a relatively larger particle size. Also, the relatively smaller particle size may have a greater water requirement to completely hydrate than a relatively larger particle size.

Energy dispersive x-ray spectroscopy is another analysis technique that may be used for measuring the physical and chemical properties of the waste materials. Energy dispersive x-ray spectroscopy is an analytical technique used to analyze the elements present in a sample and determine the chemical characterization of a sample. Other techniques may include Fourier transform infrared spectroscopy, ultraviolet-visible spectroscopy, mass spectroscopy, secondary ion mass spectrometry, electron energy mass spectrometry, dispersive x-ray spectroscopy, auger electron spectroscopy, inductively coupled plasma mass spectrometry (ICP-MS), thermal ionization mass spectroscopy, glow discharge mass spectroscopy, and x-ray photoelectron spectroscopy.

The cement components may be analyzed to determine their water requirement. Water requirement is typically defined as the amount of mixing water that is required to be added to a powdered, solid material to form a slurry of a specified consistency. Water requirement for a particular cement component may be determined by a process that includes a) preparing a Waring® blender with a specified amount of water, b) agitating the water at a specified blender rpm, c) adding the powdered solid that is being investigated to the water until a specified consistency is obtained, and d) calculating the water requirement based on the ratio of water to solids required to reach the desired consistency.

The cement components may be analyzed to determine their specific surface area. Specific surface area generally refers to the total surface area and may be reported as the total surface area per unit mass. Values obtained for specific area are dependent on the analysis technique. Any suitable analysis technique may be used, including without limitation adsorption-based methods such as Brunauer-Emmett-Teller (BET) analysis, methylene blue staining, ethylene glycol monomethyl ether adsorption, and a protein-retention method, among other.

Thermogravimetric analysis is another analysis technique that may be used for measuring the physical and chemical properties of the cement components. Thermogravimetric analysis is a method of thermal analysis wherein changes in physical and chemical properties of a sample may be measured. In general, the properties may be measured as a function of increasing temperature, such as with a constant heating rate, or as a function of time with a constant temperature or a constant mass change. Properties determined by thermogravimetric analysis may include first-order phase transitions and second-order phase transitions such as vaporization, sublimation, adsorption, desorption, absorption, chemisorption, desolvation, dehydration, oxidation and reduction reactions, ferromagnetic transition, superconducting transition, and others.

In addition to determining physical and chemical properties of the cement components themselves, laboratory tests may also be run to determine behavior of the cement components in a cement slurry. For example, the cement components may be analyzed in a cement slurry to determine their compressive strength development and mechanical properties. For example, a preselected amount of the cement component may be combined with water and lime (if needed for setting). The mechanical properties of the cement slurry may then be determined including, compressive strength, tensile strength, and Young's modulus. Any of a variety of different conditions may be used for the testing so long as the conditions are consistent for the different cement components.

Compressive strength is generally the capacity of a material or structure to withstand axially directed pushing forces. The compressive strength of the cement component may be measured at a specified time after the cement component has been mixed with water and the resultant cement slurry is maintained under specified temperature and pressure conditions. For example, compressive strength can be measured at a time in the range of about 24 to about 48 hours (or longer) after the fluid is mixed and the fluid is maintained at a temperature of from 100° F. to about 200° F. (37° C. to 93° C.) and atmospheric pressure. Compressive strength can be measured by either a destructive method or non-destructive method. The destructive method physically tests the strength of treatment fluid samples at various points in time by crushing the samples in a compression-testing machine. The compressive strength is calculated from the failure load divided by the cross-sectional area resisting the load and is reported in units of pound-force per square inch (psi). Non-destructive methods typically may employ an Ultrasonic Cement Analyzer (“UCA”), available from Fann® Instrument Company, Houston, Tex. Compressive strengths may be determined in accordance with API RP 10B-2, Recommended Practice for Testing Well Cements, First Edition, July 2005.

Tensile strength is generally the capacity of a material to withstand loads tending to elongate, as opposed to compressive strength. The tensile strength of the cement component may be measured at a specified time after the cement component has been mixed with water and the resultant cement slurry is maintained under specified temperature and pressure conditions. For example, tensile strength can be measured at a time in the range of about 24 to about 48 hours (or longer) after the fluid is mixed and the fluid is maintained at a temperature of from 100° F. to about 200° F. (37° C. to 93° C.) and atmospheric pressure. Tensile strength may be measured using any suitable method, including without limitation in accordance with the procedure described in ASTM C307. That is, specimens may be prepared in briquette molds having the appearance of dog biscuits with a one square inch cross-sectional area at the middle. Tension may then be applied at the enlarged ends of the specimens until the specimens break at the center area. The tension in pounds per square inch at which the specimen breaks is the tensile strength of the material tested.

Young's modulus also referred to as the modulus of elasticity is a measure of the relationship of an applied stress to the resultant strain. In general, a highly deformable (plastic) material will exhibit a lower modulus when the confined stress is increased. Thus, the Young's modulus is an elastic constant that demonstrates the ability of the tested material to withstand applied loads. A number of different laboratory techniques may be used to measure the Young's modulus of a treatment fluid including a cementitious component after the treatment fluid has been allowed to set for a period of time at specified temperature and pressure conditions.

Although only some select laboratory techniques may have been mentioned, it should be understood that there may many analytical techniques that may be appropriate or not appropriate for a certain sample. One of ordinary skill in the art with the benefit of this disclosure would be able to select an appropriate analytical technique to determine a certain property of interest.

Once the analytical techniques have been performed on the cement components, the data may be categorized and correlated. Some categories may include, but are not limited to, specific surface area, morphology, specific gravity, water requirement, etc. In some examples, the components may be categorized by relative amounts, including amount of at least one following: silica, alumina, iron, iron, calcium, calcium, sodium, potassium, magnesium, sulfur, oxides thereof, and combinations thereof. For example, the components may be categorized based on an oxide analysis that includes without limitation, silica content, calcium oxide content, and alumina content among other oxides that may be present in the cement component. In addition, correlations between the cement components may be generated based on the data or categorization of the data. Additionally, correlations may be defined or generated between properties of the cement components based on the data. For example, the various categories of properties may be plotted against one another. In some examples, water requirement versus specific surface area may be plotted. Accordingly, the water requirement of the cement component may be correlated to the specific surface area so that the specific surface area is a function of water requirement. Specific surface area may be used to predict reactivity of a cement component (or components). However, specific surface area may not always be available for each material as specific surface area analysis typically requires a specialized instrument. Accordingly, if the water requirement may be obtained for the cement component, the correlation between water requirement and specific surface area may be used to obtain an estimate for specific surface area, which may then be used to predict reactivity. In addition to correlations between specific surface area and reactivity, correlations may also be made between specific surface area and other mechanical properties such as tensile strength and Young's modulus.

Some cement components that are alkali soluble may include reclaimed or natural materials. Specifically, silica containing cement components may include materials such as mined materials, for example natural glass, waste materials, such as fly ash and CKD, and agricultural ashes as previously described. In some examples, the cement component that is alkali soluble may have synergistic effects with a Portland cement while others may be incompatible. In some examples, a cement component that is alkali soluble may cause gelation, high heat generation, water retention, among other effects. These and other effects may be realized during laboratory testing of the cement component in a cement slurry including Portland cement. Laboratory equipment may be configured to detect the effects of the cement component on the slurry. In some examples, equipment such a calorimeter may measure and quantify the amount of heat generation per unit mass of the cement component. Viscometers may measure the increase in gelation caused by the cement component. Each of the physical effects caused by the addition of the cement component may be measured at several concentrations and then categorized, e.g., plotted or mapped. Once a component is mapped, the effect of adding the component to a cement slurry may be predicted by referencing the categorization.

As mentioned previously, some cement components that are alkali soluble may induce gelling when included in a cement slurry. Although a higher gelling rate may be undesirable in some examples, in other examples, a higher gelling rate may be advantageous or necessary to meet the engineering design criteria. Usually one of ordinary skill in the art would select a suitable gelling agent or viscosifier for use in the cement slurry. With the benefit of mapping, one of ordinary skill would be able to select a cement component that is alkali soluble that may serve a dual purpose. For example, a cement component may increase the compressive strength of a cement slurry but also increase the gelling during mixing. If the engineering design criteria requires a higher gelling during mixing, it may be advantageous to include the cement component that increases the compressive strength while also increasing gelling. The inclusion of a cement component that exhibits multiple effects may reduce the amount of additional additives, such as gelling agents or viscosifiers, needed, which may result in complicated cement designs. Since the component's gelling effect may have been documented in a map, the amount of component to include in a cement slurry may be readily determined.

Another potentially advantageous physical effect that may be mapped is dispersing ability. Some cement components may include relatively spherical particles. The relatively spherical particles may exert a “roller bearing” effect in a cement slurry with water. The effect may cause the other components in the cement slurry to become more mobile thereby dispersing the components in the cement slurry. If particles that are roughly 1/7^{th} or smaller than the primary component in a slurry, then the apparent viscosity may decrease. Another potentially advantageous physical property that may be mapped is surface area. Surface area may relate to density wherein a relatively higher surface area particle may lower the density of a cement slurry. Particles which lower the density may be used as a low density additive. Another potentially advantageous effect that may be mapped is particle size. Components with relatively smaller particle sizes may have the ability to form a filter cake against a formation thereby blocking cement from escaping into a formation. Cement components with a small particle size may be used as a fluid loss control agent. With the benefit of the present disclosure, one of ordinary skill would be able to select a cement component and map its properties. One of ordinary skill would also be able to select a secondary property of interest of the cement component and with the benefit of the map, create a slurry with the desired properties.

Once the data is collected by the chosen laboratory techniques, categorized, and mapped, several operations may be performed on the data in order to yield predictions about a cement slurry that includes mapped cement components. Set properties, for example, may be estimated. A method of estimating the material reactivity based on the reactive index will be described below. Material reactivity may be based on many parameters such as specific surface area and specific gravity, among others. Another use for the mapped data may be to increase cement slurry performance based on parameters such as particle shape, particle size, and particle reactivity. The data may also be used to predict and capture slurry density dependence of compressive strength and use the insight gathered to design improved cement formulations. The data may also be used to predict a slurry to achieve an improved cement formulation. The criteria for just right may be compressive strength, number of components in a slurry, rheology, mechanical properties, fluid loss control properties, thickening times, and others.

Reactivity mapping may be used to estimate various mechanical properties of a cement component, including compressive strength, tensile strength, and Young's modulus. As previously described, correlations may be made between specific surface area and certain mechanical properties, such as reactivity, tensile strength, and Young's modulus. Using these correlations, the mechanical properties for a cement component or combination of cement components may be predicted.

The cement slurries described herein may include water and at least one cement component. The cement slurries may have a density suitable for a particular application. The cement slurries may have any suitable density, including, but not limited to, in the range of about 8 pounds per gallon (“ppg”) (959 kg/m³) to about 20 ppg (2397 kg/m³). The water used in the cement slurries may include, for example, freshwater, saltwater (e.g., water containing one or more salts dissolved therein), brine (e.g., saturated saltwater produced from subterranean formations), seawater, or combinations thereof. Generally, the water may be from any source, provided that it does not contain an excess of compounds that may undesirably affect other components in the cement slurry. The water may be included in an amount sufficient to form a pumpable slurry. The water may be included in the cement slurries in any suitable range, including, but not limited to, in the range of about 40% to about 200% by weight of the cement component or components (“bwoc”). By weight of cement refers to the total weight of all cement components included in the cement slurry. In some examples, the water may be included in an amount in the range of about 40% to about 150% bwoc.

The cement slurry may include a hydraulic cement. A variety of hydraulic cements may be utilized in accordance with the present disclosure, including, but not limited to, those including calcium, aluminum, silicon, oxygen, iron, and/or sulfur, which set and harden by reaction with water. Suitable hydraulic cements may include Portland cements, gypsum, and calcium aluminate cements, among others. Portland cements may be classified as Classes A, C, G, and H cements. In addition, in some examples, cements suitable for use may be classified as ASTM Type I, II, or III. Where present, the hydraulic cement generally may be included in the cement slurries in an amount sufficient to provide the desired compressive strength and/or density. The hydraulic cement may be present in the cement slurries in any suitable amount, including, but not limited to, in the range of about 0% to about 99% bwoc. In some examples the hydraulic cement may be present in an amount ranging between any of and/or including any of about 1%, about 5%, about 10%, about 20%, about 40%, about 60%, about 80%, or about 90% bwoc. In addition, the cement slurries may also be designed that are free (or essentially free) of Portland cement. Those of ordinary skill in the art, with the benefit of this disclosure, would be able to select an appropriate amount of hydraulic cement for a particular application.

The cement slurry may include a geopolymer cement, which may include an aluminosilicate source, a metal silicate source, and an activator. The geopolymer cement may react to form a geopolymer. A geopolymer is an inorganic polymer that forms long-range, covalently bonded, non-crystalline networks. Geopolymers may be formed by chemical dissolution and subsequent re-condensation of various aluminosilicates and silicates to form a 3D-network or three-dimensional mineral polymer. The activator for the geopolymer cement may include, but is not limited to, metal hydroxides, chloride salts such as KCl, CaCl₂, NaCl, carbonates such as Na₂CO₃, silicates such as sodium silicate, aluminates such as sodium aluminate, and ammonium hydroxide. The aluminosilicate source for the geopolymer cement may include any suitable aluminosilicate.

Aluminosilicate is a mineral including aluminum, silicon, and oxygen, plus counter-cations. There are potentially hundreds of suitable minerals that may be an aluminosilicate source in that they may include aluminosilicate minerals. The metal silicate source may include any suitable metal silicate. A silicate is a compound containing an anionic silicon compound. Some examples of a silicate include the orthosilicate anion also known as silicon tetroxide anion, SiO₄⁴⁻ as well as hexafluorosilicate [SiF₆]²⁻. Other common silicates include cyclic and single chain silicates which may have the general formula [SiO₂₊ₙ]²ⁿ⁻ and sheet-forming silicates ([SiO_{2.5}]⁻)ₙ. Each silicate example may have one or more metal cations associated with each silicate molecule. Some suitable metal silicate sources and may include, without limitation, sodium silicate, magnesium silicate, and potassium silicate. Where present, the geopolymer cement generally may be included in the cement slurries in an amount sufficient to provide the desired compressive strength and/or density. The geopolymer cement may be present in the cement slurries in any suitable amount, including, but not limited to, an amount in the range of about 0% to about 99% bwoc. In some examples the geopolymer cement may be present in an amount ranging between any of and/or including any of about 1%, about 5%, about 10%, about 20%, about 40%, about 60%, about 80%, or about 90% bwoc. One of ordinary skill in the art, with the benefit of this disclosure, would be able to select an appropriate amount of geopolymer cement for a particular application.

The cement slurries may include a silica source. Silica may also be referred to as silicon dioxide (SiO₂). By inclusion of a silica source, a different path may be used to arrive at a similar product as from Portland cement. For example, a pozzolanic reaction may be induced wherein silicic acid (H₄SiO₄) and portlandite (Ca(OH)₂) react to form a cement product (calcium silicate hydrate). If other compounds, such as, aluminate, are present in the silica source, additional reactions may occur to form additional cement products, such as calcium aluminate hydrates. Additionally, alumina (aluminum oxide Al₂O₃) may be present in the silica source. Calcium hydroxide necessary for the reaction may be provide from other cement components, such as Portland cement, or may be separately added to the cement slurry. Examples of suitable silica sources may include fly ash, slag, silica fume, crystalline silica, silica flour, cement kiln dust (“CKD”), natural glass, metakaolin, diatomaceous earth, zeolite, shale, and agricultural waste ash (e.g., rice husk ash, sugar cane ash, and bagasse ash), among other. Where present, the silica source generally may be included in the cement slurries in an amount sufficient to provide the desired compressive strength and/or density. The silica source may be present in the cement slurries in any suitable amount, including, but not limited to an amount in the range of about 0% to about 99% bwoc. In some examples the silica source may be present in an amount ranging between any of and/or including any of about 1%, about 5%, about 10%, about 20%, about 40%, about 60%, about 80%, or about 90% bwoc. Those of ordinary skill in the art, with the benefit of this disclosure, would be able to select an appropriate amount of silica source for a particular application.

The cement slurries may include fly ash. A variety of fly ash may be suitable, including fly ash classified as Class C and Class F fly ash according to American Petroleum Institute, API Specification for Materials and Testing for Well Cements, API Specification 10, Fifth Ed., Jul. 1, 1990. Class C fly ash includes both silica and lime, so it may set to form a hardened mass upon mixing with water. Class F fly ash generally does not contain a sufficient amount of lime to induce a cementitious reaction, therefore, an additional source of calcium ions is necessary for a set-delayed cement slurry including Class F fly ash. In some embodiments, lime may be mixed with Class F fly ash in an amount in the range of about 0.1% to about 100% by weight of the fly ash. In some instances, the lime may be hydrated lime. The fly ash may be present in the cement slurries in any suitable amount, including, but not limited to an amount in the range of about 0% to about 99% bwoc. In some examples the fly ash may be present in an amount ranging between any of and/or including any of about 1%, about 5%, about 10%, about 20%, about 40%, about 60%, about 80%, or about 90% bwoc. Those of ordinary skill in the art, with the benefit of this disclosure, would be able to select an appropriate amount of fly ash for a particular application.

The cement slurries may include slag. Slag is generally a by-product in the production of various metals from their corresponding ores. By way of example, the production of cast iron can produce slag as a granulated, blast furnace by-product with the slag generally including the oxidized impurities found in iron ore. Slag generally does not contain sufficient basic material, so slag cement may be used that further may include a base to produce a settable slurry that may react with water to set to form a hardened mass. Examples of suitable sources of bases include, but are not limited to, sodium hydroxide, sodium bicarbonate, sodium carbonate, lime, and combinations thereof. The slag may be present in the cement slurries in any suitable amount, including, but not limited to an amount in the range of about 0% to about 99% bwoc. In some examples the slag may be present in an amount ranging between any of and/or including any of about 1%, about 5%, about 10%, about 20%, about 40%, about 60%, about 80%, or about 90% bwoc. Those of ordinary skill in the art, with the benefit of this disclosure, would be able to select an appropriate amount of slag for a particular application.

The cement slurries may include cement kin dust or “CKD.” CKD refers to a partially calcined kiln feed which is removed from the gas stream and collected, for example, in a dust collector during the manufacture of cement. Usually, large quantities of CKD are collected in the production of cement that are commonly disposed of as waste. The CKD may be present in the cement slurries in any suitable amount, including, but not limited to an amount in the range of about 0% to about 99% bwoc. In some examples the CKD may be present in an amount ranging between any of and/or including any of about 1%, about 5%, about 10%, about 20%, about 40%, about 60%, about 80%, or about 90% bwoc. Those of ordinary skill in the art, with the benefit of this disclosure, would be able to select an appropriate amount of CKD for a particular application.

The cement slurries may include natural glasses. Natural glasses may exhibit cementitious properties, in that it may set and harden in the presence of hydrated lime and water. Natural glasses be present in the cement slurries in any suitable amount, including, but not limited to an amount in the range of about 0% to about 99% bwoc. In some examples the natural glasses may be present in an amount ranging between any of and/or including any of about 1%, about 5%, about 10%, about 20%, about 40%, about 60%, about 80%, or about 90% bwoc. Those of ordinary skill in the art, with the benefit of this disclosure, would be able to select an appropriate amount of silica source for a particular application.

Clays may be included in the cement slurries. Some clays may include shale or metakaolin. Among other things, clays included in the cement slurries may react with excess lime to form a suitable cementing material, for example, calcium silicate hydrate. A variety of clays are suitable, including those including silicon, aluminum, calcium, and/or magnesium. An example of a suitable shale includes vitrified shale. Zeolites may also be included in the cement slurries. Zeolites generally are porous alumino-silicate minerals that may be either a natural or synthetic material. Synthetic zeolites are based on the same type of structural cell as natural zeolites and may include aluminosilicate hydrates. As used herein, the term “zeolite” refers to all natural and synthetic forms of zeolite. Examples of zeolites may include, without limitation, mordenite, zsm-5, zeolite x, zeolite y, zeolite a, etc. Furthermore, examples including zeolite may include zeolite in combination with a cation such as Na⁺, K⁺, Ca²⁺, Mg²⁺, etc. Zeolites including cations such as sodium may also provide additional cation sources to the cement slurry as the zeolites dissolve. The clays and zeolites may be present in the cement slurries in any suitable amount, including, but not limited to an amount in the range of about 0% to about 99% bwoc. In some examples the clays and zeolites may be present in an amount ranging between any of and/or including any of about 1%, about 5%, about 10%, about 20%, about 40%, about 60%, about 80%, or about 90% bwoc. Those of ordinary skill in the art, with the benefit of this disclosure, would be able to select an appropriate amount of clays and/or zeolite for a particular application.

The cement slurries may further include hydrated lime or calcium hydroxide. In some examples, the hydrated lime may be provided as quicklime (calcium oxide) which hydrates when mixed with water to form the hydrated lime. The hydrated lime may be included in examples of the cement slurries. Where present, the hydrated lime may be included in the cement slurries in an amount in the range of from about 10% to about 100% by weight of the silica source, for example. In some examples, the hydrated lime may be present in an amount ranging between any of and/or including any of about 10%, about 20%, about 40%, about 60%, about 80%, or about 100% by weight of the silica source. One of ordinary skill in the art, with the benefit of this disclosure, would recognize the appropriate amount of hydrated lime to include for a chosen application.

In some examples, the cement slurries may include a calcium source other than hydrated lime. In general, calcium and a high pH, for example a pH of 7.0 or greater, may be needed for certain cementitious reactions to occur. A potential advantage of hydrated lime may be that calcium ions and hydroxide ions are supplied in the same molecule. In another example, the calcium source may be Ca(NO₃)₂ or CaCl₂ with the hydroxide being supplied form NaOH or KOH, for example. One of ordinary skill would understand the alternate calcium source and hydroxide source may be included in a cement slurry in the same way as hydrated lime. For example, the calcium source and hydroxide source may be included in a silica source-to-hydrated-lime weight ratio of about 10:1 to about 1:1 or a ratio of about 3:1 to about 5:1. Where present, the alternate calcium source and hydroxide source may be included in the cement slurries in an amount in the range of from about 10% to about 100% by weight of the silica source, for example. In some examples, the alternate calcium source and hydroxide source may be present in an amount ranging between any of and/or including any of about 10%, about 20%, about 40%, about 60%, about 80%, or about 100% by weight of the silica source. One of ordinary skill in the art, with the benefit of this disclosure, would recognize the appropriate amount of alternate calcium source and hydroxide source to include for a chosen application.

The cement slurries may include cement additives that may impart desirable properties to the cementing slurry. Examples of such additives include, but are not limited to: weighting agents, retarders, accelerators, activators, gas control additives, lightweight additives, gas-generating additives, mechanical-property-enhancing additives, lost-circulation materials, filtration-control additives, fluid-loss-control additives, defoaming agents, foaming agents, dispersants, thixotropic additives, suspending agents, and combinations thereof. One of ordinary skill in the art, with the benefit of this disclosure, would be able to select an appropriate additive for a particular application.

As will be appreciated by those of ordinary skill in the art, the cement slurries disclosed herein may be used in a variety of subterranean applications, including primary and remedial cementing. The cement slurries may be introduced into a subterranean formation and allowed to set. In primary cementing applications, for example, the cement slurries may be introduced into the annular space between a conduit located in a wellbore and the walls of the wellbore (and/or a larger conduit in the wellbore), wherein the wellbore penetrates the subterranean formation. The cement slurry may be allowed to set in the annular space to form an annular sheath of hardened cement. The cement slurry may form a barrier that prevents the migration of fluids in the wellbore. The cement slurry may also, for example, support the conduit in the wellbore. In remedial cementing applications, the cement slurries may be used, for example, in squeeze cementing operations or in the placement of cement plugs. By way of example, the cement slurries may be placed in a wellbore to plug an opening (e.g., a void or crack) in the formation, in a gravel pack, in the conduit, in the cement sheath, and/or between the cement sheath and the conduit (e.g., a micro annulus).

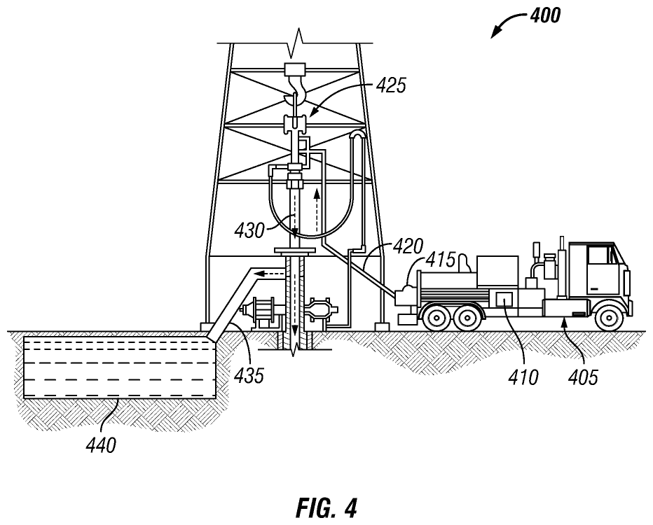

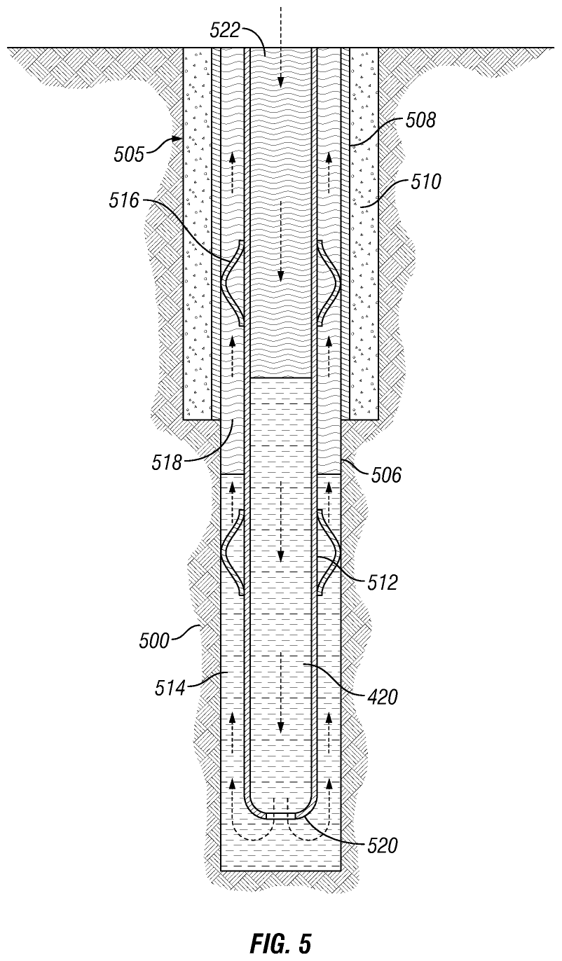

An example primary cementing technique using a cement slurry will now be described with reference to FIGS. 4 and 5. FIG. 4 illustrates surface equipment 400 that may be used in the placement of a cement slurry in accordance with certain examples. It should be noted that while FIG. 4 generally depicts a land-based operation, those skilled in the art will readily recognize that the principles described herein are equally applicable to subsea operations that employ floating or sea-based platforms and rigs, without departing from the scope of the disclosure. As illustrated by FIG. 4, the surface equipment 400 may include a cementing unit 405, which may include one or more cement trucks. The cementing unit 405 may include mixing equipment 410 and pumping equipment 41 as will be apparent to those of ordinary skill in the art. Cementing unit 405, or multiple cementing units 405, may pump a cement slurry 430 through a feed pipe 420 and to a cementing head 425 which conveys the cement slurry 430 downhole. Cement slurry 430 may displace other fluids present in the wellbore, such as drilling fluids and spacer fluids, which may exit the wellbore through an annulus and flow through pipe 435 to mud pit 440.

FIG. 5 generally depicts the placement of cement slurry 420 into a subterranean formation 500 in accordance with example examples. As illustrated, a wellbore 505 may be drilled into the subterranean formation 500. While wellbore 505 is shown extending generally vertically into the subterranean formation 500, the principles described herein are also applicable to wellbores that extend at an angle through the subterranean formation 500, such as horizontal and slanted wellbores. As illustrated, the wellbore 505 includes walls 506. In the illustrated example, a surface casing 508 has been inserted into the wellbore 505. The surface casing 508 may be cemented in the wellbore 505 by a cement sheath 510. In alternative examples, surface casing 508 may be secured in the wellbore 505 by a hardened resin or hardened resin-cement composite sheath in place of cement sheath 510. In the illustrated example, one or more additional conduits (e.g., intermediate casing, production casing, liners, etc.), shown here as casing 512 may also be disposed in the wellbore 505. As illustrated, there is a wellbore annulus 514 formed between the casing 512 and the walls 506 of the wellbore 505 and/or the surface casing 508. One or more centralizers 516 may be attached to the casing 512, for example, to centralize the casing 512 in the wellbore 505 prior to and during the cementing operation.

With continued reference to FIG. 5, a first spacer fluid 518 may be pumped down the interior of the casing 512. The first spacer fluid 518 may be allowed to flow down the interior of the casing 512 through the casing shoe 520 at the bottom of the casing 512 and up around the casing 512 into the wellbore annulus 514. After the first spacer fluid 518 has been pumped into the casing 512, cement slurry 240 may be pumped into the casing 512. In a manner similar to pumping the first spacer fluid 518, the cement slurry 420 may be allowed to flow down the interior of the casing 512 through the casing shoe 520 at the bottom of the casing 512 and up around the casing 512 into the wellbore annulus 514. After the cement slurry 420 has been pumped into the casing 512, a second spacer fluid 522 may be pumped into casing 512 and allowed to flow down the interior of the casing 512. The first spacer fluid 518 and the second spacer fluid 522 may be used to separate the cement slurry 420 from fluids introduced into the wellbore 505 either in front of or behind the cement slurry 420. Once the cement slurry 420 has been placed into the desired position in the wellbore annulus 514, the cement slurry 420 may be allowed to set in the wellbore annulus 514, for example, to form a hardened resin sheath that supports and positions the casing 512 in the wellbore 505. Alternatively, one or no spacer fluids may be used, and cement slurry 420 may not need to be separated from other fluids introduced previously or subsequently into wellbore 505. While not illustrated, other techniques may also be utilized for introduction of the cement slurry 420. By way of example, reverse circulation techniques may be used that include introducing the cement slurry 420 into the subterranean formation 500 by way of the wellbore annulus 514 instead of through the casing 512. These techniques may also utilize a first spacer fluid 518 and a second spacer fluid 522, or they may utilize one or none spacer fluids. As it is introduced, the cement slurry 420 may displace the first spacer fluid 518. At least a portion of the first spacer fluid 518 may exit the wellbore annulus 514 via a flow line 38 and be deposited, for example, in one or more mud pits 440, as shown on FIG. 4.

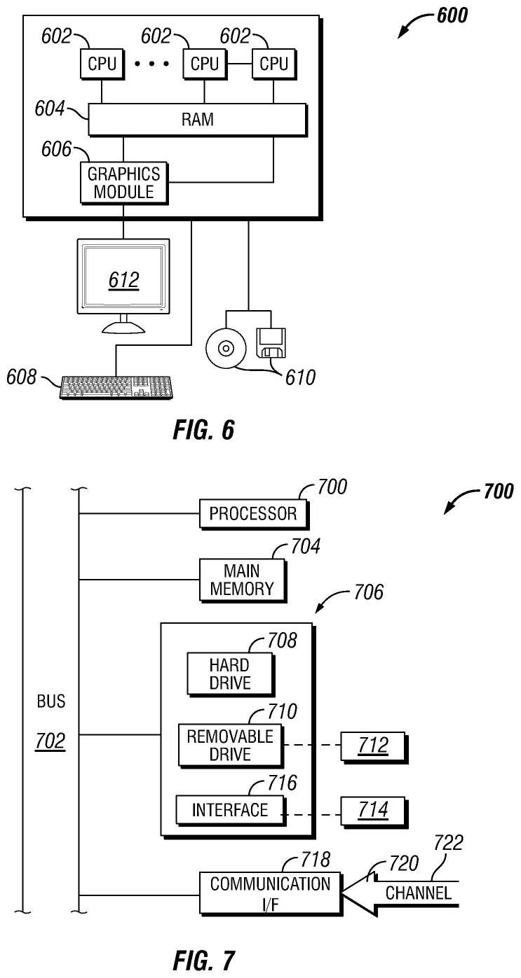

FIG. 6 generally illustrates an example of an information handling system 600 may include any instrumentality or aggregate of instrumentalities operable to compute, estimate, classify, process, transmit, receive, retrieve, originate, switch, store, display, manifest, detect, record, reproduce, handle, or utilize any form of information, intelligence, or data for business, scientific, control, or other purposes. For example, an information handling system 600 may be a personal computer, a network storage device, or any other suitable device and may vary in size, shape, performance, functionality, and price. In examples, information handling system 600 may be referred to as a supercomputer or a graphics supercomputer.

As illustrated, information handling system 600 may include one or more central processing units (CPU) or processors 602. Information handling system 600 may also include a random-access memory (RAM) 604 that may be accessed by processors 602. It should be noted information handling system 600 may further include hardware or software logic, ROM, and/or any other type of nonvolatile memory. Information handling system 600 may include one or more graphics modules 606 that may access RAM 704. Graphics modules 606 may execute the functions carried out by a Graphics Processing Module (not illustrated), using hardware (such as specialized graphics processors) or a combination of hardware and software. A user input device 608 may allow a user to control and input information to information handling system 600. Additional components of the information handling system 600 may include one or more disk drives, output devices 612, such as a video display, and one or more network ports for communication with external devices as well as a user input device 608 (e.g., keyboard, mouse, etc.). Information handling system 600 may also include one or more buses operable to transmit communications between the various hardware components.

Alternatively, systems and methods of the present disclosure may be implemented, at least in part, with non-transitory computer-readable media. Non-transitory computer-readable media may include any instrumentality or aggregation of instrumentalities that may retain data and/or instructions for a period of time. Non-transitory computer-readable media may include, for example, storage media 610 such as a direct access storage device (e.g., a hard disk drive or floppy disk drive), a sequential access storage device (e.g., a tape disk drive), compact disk, CD-ROM, DVD, RAM, ROM, electrically erasable programmable read-only memory (EEPROM), and/or flash memory; as well as communications media such wires, optical fibers, microwaves, radio waves, and other electromagnetic and/or optical carriers; and/or any combination of the foregoing.

FIG. 7 illustrates additional detail of information handling system 600. For example, information handling system 600 may include one or more processors, such as processor 700. Processor 700 may be connected to a communication bus 702. Various software embodiments are described in terms of this exemplary computer system. After reading this description, it will become apparent to a person skilled in the relevant art how to implement the example embodiments using other computer systems and/or computer architectures.

Information handling system 600 may also include a main memory 704, preferably random-access memory (RAM), and may also include a secondary memory 706. Secondary memory 706 may include, for example, a hard disk drive 708 and/or a removable storage drive 710, representing a floppy disk drive, a magnetic tape drive, an optical disk drive, etc. Removable storage drive 710 may read from and/or writes to a removable storage unit 712 in any suitable manner. Removable storage unit 712, represents a floppy disk, magnetic tape, optical disk, etc. which is read by and written to by removable storage drive 710. As will be appreciated, removable storage unit 712 includes a computer usable storage medium having stored therein computer software and/or data.

In alternative embodiments, secondary memory 706 may include other operations for allowing computer programs or other instructions to be loaded into information handling system 600. For example, a removable storage unit 714 and an interface 716. Examples of such may include a program cartridge and cartridge interface (such as that found in video game devices), a removable memory chip (such as an EPROM, or PROM) and associated socket, and other removable storage units 714 and interfaces 716 which may allow software and data to be transferred from removable storage unit 714 to information handling system 600.

In examples, information handling system 600 may also include a communications interface 718. Communications interface 718 may allow software and data to be transferred between information handling system 600 and external devices. Examples of communications interface 718 may include a modem, a network interface (such as an Ethernet card), a communications port, a PCMCIA slot and card, etc. Software and data transferred via communications interface 718 are in the form of signals 720 that may be electronic, electromagnetic, optical or other signals capable of being received by communications interface 718. Signals 720 may be provided to communications interface via a channel 722. Channel 722 carries signals 720 and may be implemented using wire or cable, fiber optics, a phone line, a cellular phone link, an RF link and/or any other suitable communications channels. For example, information handling system 600 includes at least one memory 704 operable to store computer-executable instructions, at least one communications interface 702, 718 to access the at least one memory 704; and at least one processor 700 configured to access the at least one memory 704 via the at least one communications interface 702, 718 and execute computer-executable instructions.

In this document, the terms “computer program medium” and “computer usable medium” are used to generally refer to media such as removable storage unit 712, a hard disk installed in hard disk drive 708, and signals 720. These computer program products may provide software to computer system 600.

Computer programs (also called computer control logic) may be stored in main memory 804 and/or secondary memory 706. Computer programs may also be received via communications interface 718. Such computer programs, when executed, enable information handling system 600 to perform the features of the example embodiments as discussed herein. In particular, the computer programs, when executed, enable processor 700 to perform the features of the example embodiments. Accordingly, such computer programs represent controllers of information handling system 600.

In examples with software implementation, the software may be stored in a computer program product and loaded into information handling system 600 using removable storage drive 710, hard disk drive 708 or communications interface 718. The control logic (software), when executed by processor 700, causes processor 700 to perform the functions of the example embodiments as described herein.

In examples with hardware implementation, hardware components such as application specific integrated circuits (ASICs). Implementation of such a hardware state machine so as to perform the functions described herein will be apparent to persons skilled in the relevant art(s). It should be noted that the disclosure may be implemented at least partially on both hardware and software.

The methods described herein may be carried out, at least in part, using a computer system including a computer-accessible medium, the computer-accessible medium containing a computer program that causes a processor to execute instructions that carry out at least some of the method steps described herein. In general, a computer-accessible medium may include any tangible or non-transitory storage media or memory media such as electronic, magnetic, or optical media—e.g., disk or CD/DVD-ROM coupled to the computer. The terms “tangible” and “non-transitory,” as used herein, are intended to describe a computer-readable storage medium (or “memory”) excluding propagating electromagnetic signals, but are not intended to otherwise limit the type of physical computer-readable storage device that is encompassed by the phrase computer-readable medium or memory. For instance, the terms “non-transitory computer-readable medium” or “tangible memory” are intended to encompass types of storage devices that do not necessarily store information permanently, including for example, random access memory (RAM), flash memory, or other volatile memory types. Program instructions and data stored on a tangible computer-accessible storage medium in non-transitory form may further be transmitted by transmission media or signals such as electrical, electromagnetic, or digital signals, which may be conveyed via a communication medium such as a network and/or a wireless link.

The following statements may include some embodiments of the disclosure but should not be read to be limiting to any particular embodiment.

Statement 1. A method comprising: providing a model of cement temperature sensitivity; designing a cement composition, based at least partially on the model of cement temperature sensitivity; and preparing the cement composition.

Statement 2. The method of statement 1 wherein the model of temperature sensitivity includes a model of activation energy, and wherein the model of activation energy includes a function of physicochemical parameters, a model of extent of hydration, and a model of effective time.

Statement 3. The method of any of statements 1-2 wherein the model of temperature sensitivity includes a model of activation energy derived from correlating calorimetric data to an activation energy.

Statement 4. The method of any of statements 1-3 wherein the model of temperature sensitivity includes a model of extent of hydration in the form of:

$$H = {H}_{u} {e}^{- {( \frac{τ}{{t}_{e}} )}^{β}}$$

where H is extent of hydration, Hᵤ is ultimate extent of hydration, to is effective time, and τ and β are kinetic rate parameters.

Statement 5. The method of any of statements 1-4 wherein the model of temperature sensitivity includes a model of effective time in the form of:

$$\frac{\partial {t}_{e}}{\partial t} = exp ( \frac{E}{R} ( \frac{1}{{T}_{ref}} - \frac{1}{T} ) )$$

where E is activation energy, R is a gas constant, T_{ref} is a reference temperature, and T is a current temperature.

Statement 6. The method of any of statements 1-5 further comprising: modifying the cement composition to produce a modified cement composition if a predicted compressive strength from the model of cement temperature sensitivity does not meet or exceed a time dependent compressive strength requirement; calculating a predicted compressive strength of the modified cement composition using the model of cement temperature sensitivity; comparing the compressive strength of the modified cement composition to a time dependent compressive strength requirement; and preparing the modified cement composition if the predicted compressive strength meets or exceeds the time dependent compressive strength requirement.

Statement 7. The method of any of statements 1-6 further comprising iteratively modifying the cement composition until the compressive strength of the modified cement composition meets or exceeds the time dependent compressive strength requirement.

Statement 8. The method of any of statements 1-7 wherein iteratively modifying the cement composition includes modifying a concentration of the water, a concentration of at least one cementitious component, or both.

Statement 9. A method comprising: providing a plurality of cementitious components; providing a design parameter, a downhole temperature, and model of cement temperature sensitivity wherein the model of cement temperature sensitivity includes a function of physicochemical parameters about the cementitious components, a model of extent of hydration, a model of effective time, a model of activation energy; generating a cement composition, wherein the cement composition includes cementitious components selected from the plurality of cementitious components; calculating a predicted design parameter of the cement composition using the model cement temperature sensitivity; comparing the predicted design parameter of the cement composition to the design parameter; and preparing the cement composition if the predicted design parameter meets or exceeds the design parameter.

Statement 10. The method of statement 9 wherein the model of activation energy is a regression model of activation energy and physicochemical data.

Statement 11. The method of any of statements 9-10 wherein the model of activation energy is derived from correlating calorimetric data to an activation energy.

Statement 12. The method of any of statements 9-11 wherein the model of extent of hydration is in the form of:

$$H = {H}_{u} {e}^{- {( \frac{τ}{{t}_{e}} )}^{β}}$$

where H is extent of hydration, Hᵤ is ultimate extent of hydration, tₑ is effective time, and τ and β are kinetic rate parameters.

Statement 13. The method of any of statements 9-12 wherein the model of effective time is in the form of:

$$\frac{\partial {t}_{e}}{\partial t} = exp ( \frac{E}{R} ( \frac{1}{{T}_{ref}} - \frac{1}{T} ) )$$

where E is activation energy, R is a gas constant, T_{ref} is a reference temperature, and T is a current temperature.

Statement 14. A non-transitory computer readable medium having data stored therein representing software executable by a computer, the software including instructions comprising: instructions to generate a design of a cement composition comprising at least one of a plurality of cementitious components based on a model of cement temperature sensitivity.

Statement 15. The non-transitory computer readable medium of statement 14 wherein the model of cement temperature sensitivity includes a function of physicochemical parameters about the cementitious components, a model of extent of hydration, a model of effective time, and a model of activation energy.

Statement 16. The non-transitory computer readable medium of any of statements 14-15 wherein the model of activation energy is a regression model of activation energy and physicochemical data.

Statement 17. The non-transitory computer readable medium of any of statements 14-16 further comprising instructions to accept a downhole temperature.

Statement 18. The non-transitory computer readable medium of any of statements 14-17 wherein the instructions to generate the design of the cement composition includes instruction to generate the cement composition based at least in part on the downhole temperature.

Statement 19. The non-transitory computer readable medium of any of statements 14-18 wherein the model of extent of hydration is in the form of:

$$H = {H}_{u} {e}^{- {( \frac{τ}{{t}_{e}} )}^{β}}$$

where H is extent of hydration, Hᵤ is ultimate extent of hydration, to is effective time, and τ and β are kinetic rate parameters.

Statement 20. The non-transitory computer readable medium of statement 15 wherein the model of effective time is in the form of:

$$\frac{\partial {t}_{e}}{\partial t} = exp ( \frac{E}{R} ( \frac{1}{{T}_{ref}} - \frac{1}{T} ) )$$

where E is activation energy, R is a gas constant, T_{ref} is a reference temperature, and T is a current temperature.

The disclosed cement slurries and associated methods may directly or indirectly affect any pumping systems, which representatively includes any conduits, pipelines, trucks, tubulars, and/or pipes which may be coupled to the pump and/or any pumping systems and may be used to fluidically convey the cement slurries downhole, any pumps, compressors, or motors (e.g., topside or downhole) used to drive the cement slurries into motion, any valves or related joints used to regulate the pressure or flow rate of the cement slurries, and any sensors (i.e., pressure, temperature, flow rate, etc.), gauges, and/or combinations thereof, and the like. The cement slurries may also directly or indirectly affect any mixing hoppers and retention pits and their assorted variations.

It should be understood that the slurries and methods are described in terms of “comprising,” “containing,” or “including” various components or steps, the slurries and methods can also “consist essentially of” or “consist of” the various components and steps. Moreover, the indefinite articles “a” or “an,” as used in the claims, are defined herein to mean one or more than one of the elements that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular examples disclosed above are illustrative only, as the present disclosure may be modified and practiced in different but equivalent manners apparent to those skilled in the art having the benefit of the teachings herein. Although individual examples are discussed, the disclosure covers all combinations of all those examples. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative examples disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

What is claimed is:

1. A method comprising:
providing a model of cement temperature sensitivity;
designing a cement composition, based at least partially on the model of cement temperature sensitivity; and
preparing the cement composition.

2. The method of claim 1 wherein the model of temperature sensitivity comprises a model of activation energy, and wherein the model of activation energy comprises a function of physicochemical parameters, a model of extent of hydration, and a model of effective time.

3. The method of claim 1 wherein the model of temperature sensitivity comprises a model of activation energy derived from correlating calorimetric data to an activation energy.

4. The method of claim 1 wherein the model of temperature sensitivity comprises a model of extent of hydration in the form of:
$$H = {H}_{u} {e}^{- {( \frac{τ}{{t}_{e}} )}^{β}}$$
where H is extent of hydration, Hᵤ is ultimate extent of hydration, tₑ is effective time, and τ and β are kinetic rate parameters.

5. The method of claim 1 wherein the model of temperature sensitivity comprises a model of effective time in the form of:
$$\frac{\partial {t}_{e}}{\partial t} = exp ( \frac{E}{R} ( \frac{1}{{T}_{ref}} - \frac{1}{T} ) )$$
where E is activation energy, R is a gas constant, T_{ref} is a reference temperature, and T is a current temperature.

6. The method of claim 1 further comprising:
modifying the cement composition to produce a modified cement composition if a predicted compressive strength from the model of cement temperature sensitivity does not meet or exceed a time dependent compressive strength requirement;
calculating a predicted compressive strength of the modified cement composition using the model of cement temperature sensitivity;
comparing the compressive strength of the modified cement composition to a time dependent compressive strength requirement; and
preparing the modified cement composition if the predicted compressive strength meets or exceeds the time dependent compressive strength requirement.

7. The method of claim 6 further comprising iteratively modifying the cement composition until the compressive strength of the modified cement composition meets or exceeds the time dependent compressive strength requirement.

8. The method of claim 7 wherein iteratively modifying the cement composition comprises modifying a concentration of the water, a concentration of at least one cementitious component, or both.

9. A method comprising:
providing a plurality of cementitious components;
providing a design parameter, a downhole temperature, and model of cement temperature sensitivity wherein the model of cement temperature sensitivity comprises a function of physicochemical parameters about the cementitious components, a model of extent of hydration, a model of effective time, and a model of activation energy;
generating a cement composition, wherein the cement composition includes cementitious components selected from the plurality of cementitious components;
calculating a predicted design parameter of the cement composition using the model cement temperature sensitivity;
comparing the predicted design parameter of the cement composition to the design parameter; and
preparing the cement composition if the predicted design parameter meets or exceeds the design parameter.

10. The method of claim 9 wherein the model of activation energy is a regression model of activation energy and physicochemical data.

11. The method of claim 10 wherein the model of activation energy is derived from correlating calorimetric data to an activation energy.

12. The method of claim 9 wherein the model of extent of hydration is in the form of:
$$H = {H}_{u} {e}^{- {( \frac{τ}{{t}_{e}} )}^{β}}$$
where H is extent of hydration, Hᵤ is ultimate extent of hydration, tₑ is effective time, and τ and β are kinetic rate parameters.

13. The method of claim 9 wherein the model of effective time is in the form of:
$$\frac{\partial {t}_{e}}{\partial t} = exp ( \frac{E}{R} ( \frac{1}{{T}_{ref}} - \frac{1}{T} ) )$$
where E is activation energy, R is a gas constant, T_{ref} is a reference temperature, and T is a current temperature.

14. A non-transitory computer readable medium having data stored therein representing software executable by a computer, the software including instructions comprising:
instructions to generate a design of a cement composition comprising at least one of a plurality of cementitious components based on a model of cement temperature sensitivity.

15. The non-transitory computer readable medium of claim 14 wherein the model of cement temperature sensitivity comprises a function of physicochemical parameters about the cementitious components, a model of extent of hydration, a model of effective time, and a model of activation energy.

16. The non-transitory computer readable medium of claim 15, wherein the model of activation energy is a regression model of activation energy and physicochemical data.

17. The non-transitory computer readable medium of claim 14 further comprising instructions to accept a downhole temperature.

18. The non-transitory computer readable medium of claim 17 wherein the instructions to generate the design of the cement composition comprises instruction to generate the cement composition based at least in part on the downhole temperature.

19. The non-transitory computer readable medium of claim 15 wherein the model of extent of hydration is in the form of:
$$H = {H}_{u} {e}^{- {( \frac{τ}{{t}_{e}} )}^{β}}$$
where H is extent of hydration, Hᵤ is ultimate extent of hydration, tₑ is effective time, and τ0 and β are kinetic rate parameters.

20. The non-transitory computer readable medium of claim 15 wherein the model of effective time is in the form of:
$$\frac{\partial {t}_{e}}{\partial t} = exp ( \frac{E}{R} ( \frac{1}{{T}_{ref}} - \frac{1}{T} ) )$$
where E is activation energy, R is a gas constant, T_{ref} is a reference temperature, and T is a current temperature.
